(19) United States
(12) Patent Application Publication
(10) Pub. No.: US 2004/0101824 A1
(43) Pub. Date: May 27, 2004

(54) Method for identifying substances which are suitable to be used as medicaments for the treating virus infections or for testing the effectiveness of such substances

(76) Inventors: Christoph Brauchle, Tutzing (DE); Georg Seisenberger, Munchen (DE); Martin Reid, Sinning (DE); Thomas Endress, Hamburg (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/332,146

(22) PCT Filed: Jul. 6, 2001
(86) PCT No.: PCT/EP01/07781

(30) Foreign Application Priority Data
Jul. 6, 2000 (DE) 100328598

(51) Int. Cl. C12Q 1/70

(52) U.S. Cl. 435/5

(57) ABSTRACT

The invention relates to a method for identifying substances which are suitable to be used as medicaments for treating virus infections or for testing the effectiveness of such substances. The invention also relates to the use of a corresponding method for observing the infection route and/or the infection mechanism of viruses in cells, especially viruses that are provided for gene transfers for gene expression or as vectors and/or for gene therapy.

The present invention relates to methods for identifying substances suitable as medicaments for the treatment of viral infections or for testing the effectiveness of said substances. The present invention further relates to the application of a corresponding method to observing the route or/and mechanism of infection of viruses in cells, in particular of viruses which are intended to be used as gene shuttles for gene expression or as vectors or/and for gene therapy.

The detection and control of viruses are important tasks in many areas, but in particular in medicine and biology. Viruses are found in areas as different as viral infection (viruses entering the living cell), immunology (viruses as antigens or antigen producers) and gene expression or gene therapy (viruses as gene shuttles). Frequently, the detection of very small virus concentrations, more or less down to the ultimate analytical detection limit, namely the detection of a single virus, is of particular interest.

Previously, viruses have been detected, for example, indirectly via their subsequent expressed products or have been radiolabeled. Attempts to detect viruses which have been labeled by fluorescent dyes previously had to be limited, for reasons of sensitivity, to detecting a large accumulation of viruses (J. S. Bartlett, R. Wilcher, R. J. Samulski, “Infectious Entry Pathway of Adeno-Associated Virus and Adeno-Associated Virus Vectors”, Journal of Virology, Vol. 74 (2000) 2777) which, in addition, had been labeled frequently not only with one dye but with a multiplicity of dyes per virus (P. L. Leopold, B. Ferris, I. Grinberg, S. Worgall, N. R. Hackett, R. G. Crystal, “Fluorescent Virions: Dynamic Tracking of the Pathway of Adenoviral Gene Transfer Vectors in Living Cells”, Human Gene Therapy 9 (1998) 367).

In the field of medicine, controlling viruses is still difficult and frequently only goes as far as stimulating and supporting the immune system. Therefore, there is still a need for medicaments which can prevent, alleviate or/and cure viral infections. One possible idea here is to attack the virus at all stages of the cycle of infection. Up until now, however, there has been a lack of specific possibilities of testing substances for their suitability for controlling viruses and for treating viral infections, in particular of determining the action or at least the site of action of a substance. For a useful identification and characterization of medicaments, however, it would be especially advantageous if the stage of the viral infection, at which a particular substance acts on the cycle, could also be determined in the process. This would make it possible to modify substances also in a targeted manner in order to further enhance or adapt their properties in this respect.

It was therefore the object of the present invention to provide a possibility of enabling the identification of medicaments for the treatment of viral infections by a method which allows detecting the site of action and the changes in the cycle of infection, generated by said action.

According to the invention, the object is achieved by a method for identifying substances suitable as medicaments for the treatment of viral infections or for testing the effectiveness of said substances, in which individual viruses are labeled with one or more fluorescent dye molecules and the influence of the substance on the viruses or/and their route of infection via the fluorescence of said dye, after excitation, in comparison with a control sample.

The method of the invention provides, for the first time, a possibility of tracking the route of a single virus in a cell and also further migration of the virus components within the cell. If this is carried out in parallel for a sample to which a substance is added whose influence on the viral infection is to be studied and for a control sample, it is possible to gain information about the question as to whether the substance influences the cycle of infection and at which site such an influence takes place. Using the method of the invention in which individual viruses are labeled with only one or with several, but a few, fluorescent dye molecules results in the following advantages:

a) the biological/physiological properties of the virus, for example virus/receptor interaction, remains more or less unchanged by attaching a single dye molecule, in contrast to attaching a multiplicity of dye molecules as is described according to Leopold (see above),

b) the experiment requires only low to very low virus concentrations, since already a single virus can be detected (working with physiologically relevant concentrations or very low and harmless concentrations is possible),

c) real time observation of a single virus with high time and spatial resolution is possible, even in the living cell.

d) Kinetic studies and statistics of secondary processes on a multiplicity of individual viruses are possible without interference by superposition effects, as they appear in an ensemble of viruses.

The method of the invention allows, for the first time, specific screening for antivirally active substances, and it is possible to postulate even a desired site or mechanism of action as a determination criterion. It is possible with the aid of the method of the invention to visualize trajectories of individual viruses, contributing to revealing mechanistic details of the cycle of infection. Thus it is possible, for the first time, to observe in detail and under physiological-conditions the individual steps of a viral infection of a living cell, such as docking to receptors on the cell membrane, penetrating the cell membrane, diffusion or active transport in the cytoplasm, or entering the nucleus and deposition of the viral DNA in the nucleus. The addition of substances to be studied and their effect on the viral infection can be observed in detail.

The method of the invention is based on recording individual viruses with the aid of the single molecule fluorescence technique.

Owing to sensitivity problems, classical fluorescence microscopy of the prior art previously only enabled visualization of high virus concentrations (10⁵ to 10⁶ viruses per cell) and/or frequently also a large number of fluorescent labels per virus. This caused the following problems:

unphysiological conditions due to 10⁵ to 10⁶ viruses per cell, causing damage to the cell;

disruption of virus-cell interaction due to the high degree of labeling of virus;

the infection could only be visualized as an accumulation of a large number of viruses with poor time and space resolution;

kinetic studies are greatly impaired by superposition effects of virus accumulation;

detection of the individual steps of infection are frequently extremely delayed,

the influence of an active substance under said limitations could be determined as an overall effect at best, locating the site of action of an active substance was not possible.

Although methods in which individual molecules are labeled have already been described, it has been impossible until now to observe the entire route of infection of a virus.

However, internalization and intracellular movement of a virus in living cells in particular are of fundamental importance in order to understand the cause of the viral infections, to design antiviral drugs and also to develop viruses as gene shuttles for gene expression and to develop vectors for gene therapy.

The method of the invention is characterized in that the individual virus is labeled with one or more fluorescent dye molecules and can be tracked via fluorescence. The method can therefore be applied to viruses for all possible uses for which it is important to achieve high to very high analytical sensitivity, in particular for detecting individual viruses.

The method for recording individual viruses makes it possible, in particular, to record viruses on their route of infection into a living cell. The virus is visible in a microscope by way of a fluorescent spot which can be tracked with a spatial resolution of from 1 to 40 nm in real time and with a time resolution of from 1 to 40 ms.

The method allows detection of

a) just a single virus

b) which is labeled with just one dye and whose surface is practically unchanged and which essentially retains its biochemical/physiological properties,

c) and thus is the ultimate detection limit for a virus in medical and biochemical analytics. This makes it possible, for example, to detect mechanisms of viruses entering the living cell with the highest analytical resolution. Likewise, said method makes it possible to test with high precision remedies/methods preventing viruses from entering the cell. Furthermore, it is possible to recognize the behavior of viruses, such as, for example, their selectivity with respect to entering particular cells, and appropriate defence measures.

d) All of this can be followed in real time with high space and time resolution, even in the living cell.

The detection sensitivity in the method of the invention is such that a single fluorescent dye molecule can already be detected. Thus, the virus needs to be labeled only with one (or several, but only a few, preferably 2 to 3) dye molecules in order to be already detectable as a single virus. The advantage of this is the minimum labeling leaves the viral surface more or less unchanged and the virus retains its biological/physiological properties.

The fluorescent dyes used in the method of the invention are bound to the virus, in a preferred embodiment to N-terminal amino acid residues of the viral capsid proteins. Binding to proteins site-specifically mutated with cysteine is also a possibility which is preferred according to the invention.

It is further possible to bind also at least two dyes which are distinguishable spectrally, by lifetime, by polarization spectroscopy or by (Förster resonance) energy transfer experiments to various viral regions. Thus it is possible, for example, to bind dye 1 to the capsid and dye 2 to the DNA of the virus. This allows, for example, separation of viral capsid and DNA to be monitored later.

In a further preferred embodiment, the dye molecule or molecules is/are bound to the viral DNA or to internal proteins.

Dye molecules may in principle be bound directly, but also via selective antibodies which are directed against a viral component and are themselves labeled with a dye molecule. In a further preferred embodiment, labeling is carried out via a specific binding pair or via cargo systems transported with the virus. Examples of such cargo systems are liposomes or polylysine-DNA complexes (E. Wagner et al. P.N.A.S. 89 (1992) 6099). In addition to DNA, such cargo systems may also comprise, for example, other medical substances.

When using a specific binding pair, a part of the virus is attached to one partner of the binding pair, with the label then being located on the other partner of said binding pair. When the two components meet, the two partners of the binding pair form a complex, and thus the virus is labeled. The binding pair used is preferably biotin/streptavidin, with biotin preferably bound to viral protein (e.g. capsid protein) and the dye preferably conjugated to streptavidin.

Within the scope of the present invention, a very simple method is described which is used to detect individual viruses labeled with a fluorescent dye with the aid of single molecule fluorescence microscopy. For this purpose, the fluorescent dye on the virus is excited, for example, with the aid of a light source (preferably a laser, preferably a HeNe laser or a laser diode) through a microscope objective (extended beam), and fluorescence is collected through the same microscope objective and detected using a highly sensitive detector (in particular a CCD camera or an avalanche photodiode) [Farfield Excitation Imaging T. Schmidt, G. J. Schutz, W. Baumgartner, H. J. Gruber, H. Schindler, J. Phys. Chem. 99 (1995) 17262)]. Other single molecule methods may also be applied for this purpose, such as, for example, Scanning Confocal Microscopy (J. J. Macklin, K. Trautman, T. D. Harris, L. E. Brus. Science 272 (1996) 255.), Total Internal Reflection Imaging/Evanescent Field (T. Funatsu, Y. Harada, M. Tokunaga, K. Saito, T. Yanagida, Nature 374 (1995) 555.), Scanning Nearfield Optical Microscopy (E. Betzig, R. J. Chichester, Science 262 (1993) 1422.), Photon Burst Detection (R. A. Keller et al. Appl. Spectrosc. 50 (1996) A12.) and Fluorescence Correlation Spectroscopy (M. Eigen, R. Rigler. Proc. Natl. Acad. Sci. U.S.A. 91 (1994) 5740.), and combinations thereof.

Fluorescent dyes suitable according to the invention are dyes with good fluorescence properties, preferably in the long-wave green or in the red spectral region, which can be excited using a large absorption cross section and which display high fluorescence quantum yield and high photostability.

According to the invention, particularly preferred dye is distinguished by high fluorescence quantum yields, high photostability, typically excitation in the green or red spectral region with high absorption cross section and attachability, for example covalently (J. S. Bartlett, R. Wilcher, R. J. Samulski,. “Infectious Entry Pathway of Adeno-Associated Virus and Adeno-Associated Virus Vectors”, Journal of Virology, Vol. 74 (2000) 2777) to a free N-terminal amino acid residue of the capsid protein of a virus. It is further possible to introduce cysteine by means of site-specific mutagenesis at a particular position of the capsid protein and to bind there the dye specifically to the SH group. It is also possible to attach, for example, two different dyes at two different positions (e.g. capsid and DNA of the virus).

Suitable according to the invention are classical organic fluorophores, and the experiments can be carried out particularly successfully using Cy5(copyright Amersham) (protected dye).

It is, however, also possible to use, in addition to such organic fluorophores, expressible fluorescent proteins such as green fluorescent protein (GFP) and its mutants or luminescent nanoparticles. Luminescent nanoparticles are known to the skilled worker and have been described in the specialist literature (Bruchez M, Moronne M, Gin P, Weiss S, Alivisatos A P, Semiconductornanocrystals as fluorescent biological labels”, Science 281 (1998) 2013-6).

The sample consists, for example, of monodisperse living cells which have grown on a slide and which are covered by a nutrient solution. At the start of the experiment, said nutrient solution is removed and replaced by a buffer solution (e.g. PBS), followed by adding the virus solution, marking the start of the experiment.

The technique applied in the method of the invention is characterized in that excitation is carried out via a light source, preferably a laser, particularly preferably a simple HeNe laser, or a laser diode. However, excitation may also be carried out within the scope of the present invention via a strong laser using “two photon excitation”.

Furthermore, preference is given to exciting two or more different fluorescent dyes simultaneously by using different laser lines and a laser with variable frequency.

The light is focussed in a microscope or microscope objective (also confocal microscope) and projected on the sample. Within the scope of the present invention, preference is again given to the microscope used comprising various modes which enable the cell and its components to be depicted simultaneously or/and independent of the detection of the virus. Thus, the cell may be depicted in the transmitted light method using a separate light source, and it is possible to use phase contrast, differential interference contrast or polarization contrast techniques.

Within the scope of the present invention, further preference is given to two- or three-dimensional imaging of a cell and that said cell or its components are depicted confocally or by methods using widefield illumination. Individual organelles or similar cellular subunits are detected in the transmitted light mode and/or via fluorescence methods using labeled components.

In the case of two-dimensional tracking of viral movement, excitation is carried out using the widefield method and the movement in axial z dimension is tracked, if necessary, by controlling the relative sample/microscope objective movement which tracks the viral movement automatically on the basis of the half-width of the fluorescence signal.

Within the scope of the present invention, preference is again given to tracking selectively labeled subunits in viruses, biomolecules functionally interacting with viruses and viruses of a subsequent generation, which have been generated by expression in the cell, separately from one another or/and determining the functional relationship of these units or molecules with one another or with cell components. Thus it is possible, for example, to follow a virus disintegrating into the components capsid and genome and also to follow a virus docking with a receptor of the cell membrane or with a nuclear pore complex of the nuclear membrane.

In this connection, it is possible to identify and characterize separately from one another sections of the infection biology of the virus, such as adsorption to receptors, endocytosis, diffusion in endosomes, free diffusion, abnormal diffusion, diffusion in corals or inclusions, active transport, for example by means of motor proteins, penetration of the nuclear membrane, colocalization with nuclear pore complexes, disintegration of the virus, insertion of the genome into cellular DNA or production of viruses of the next generation, under physiologically relevant conditions.

In a further preferred embodiment of the invention, a microscope is used which has a device for fixing the slide carrying the sample, which device is temperature-controllable. This makes it possible to carry out the method of the invention at a desired predetermined temperature. It is particularly preferred here to carry out the sample preparation at approx. 4° C., i.e. at a temperature at which there is hardly any change in the virus/cell situation, providing a good starting point for the subsequent observation. The actual observation of the viral infection is then preferably carried out at a temperature of 37° C. which in turn corresponds, to a large extent, to the physiological conditions.

The sample consists of cells, typically of cells which have grown flat (monodisperse) on the sample support. The cells are preferably covered by a nutrient solution or a buffer solution. Fluorescently labeled viruses become visible due to their fluorescence which is typically separated from the excitation light by means of filters and is detected using highly sensitive detectors, typically a CCD camera or an avalanche photodiode.

It is also possible, within the scope of the present invention, to observe the route of infection or/and the mechanism of infection of viruses in cells, and this is particularly interesting in the case of observing viruses intended to be used as vectors and/or for gene therapy. To this end, a method as described analogously for drug screening above is applied and the virus and its induced secondary products are observed. In this case, it is in turn possible to gain information about the route of infection, the efficiency of infection and the final location of the introduced nucleic acids. Of course, it is also possible here to compare control samples to which additionally further substances have been added. Substances which may be used here are also those which increase the permeability of the cell membrane or similar substances which are preferably employed when using viruses as vectors and for gene therapy. It is also possible, with the aid of the method of the invention, to screen for those substances which facilitate a viral infection and make it more efficient. The method may also be used for determining those cargo systems which can be, for example, introduced successfully into the cell or transported to particular cellular locations and attachment of such systems to the virus. It is also possible to observe possibly intentional dissolving or detaching of the cargo system and/or of its components at particular sites within the cell.

Finally, the method of the invention can also lead to designing novel medicaments, since it is possible to gain information about the effects of substances on the viral infection and to postulate novel substances having advantageous properties by means of combining the findings about particular substances. Thus it is possible to compose from substances, or parts thereof, classified as effective according to the invention further substances which exhibit synergistic effects.

In the method of the invention, it is also possible to observe the route of infection of viruses or the route of induced secondary products of viruses in cells, with the viruses, as gene shuttles, encoding expressible substances which are produced by the host cell following the viral infection.

The following example and the figures are intended to further illustrate the invention.

### Example 1

In the example of observing the route of infection of adeno-associated viruses (AAV) in HeLa cells, the following was observed:

1.) The diffusional motion of a single virus was tracked outside the cell membrane with a spatial resolution of 40 nm. As long as the virus is at a relatively large distance from the cell membrane, the diffusion coefficient observed is the same as in solution. When the virus approaches the cell membrane, diffusion slows down visibly and finally stops at the cell membrane (adsorption of the virus on the receptor).

2.) Diffusional motion of the virus in the cytoplasm of the cell after endocytosis. Observation of the diffusional motion of the endosome, which has a very much smaller diffusion coefficient than the virus in solution. Followed by the virus being released from the endosome.

3.) Virus entering the nucleus and moving in the nucleus with delivery of DNA.

In all three phases, the virus is detected as a local fluorescent spot (made visible by the attached fluorescent molecule) with a spatial resolution of approx. 40 nm.

The fluorescence microscope used is a standard fluorescence microscope. The light source used is an external laser (HeNe laser with an output power of 35 MW in the example described). In order to be able to produce very high detection efficiencies, the use of an inverted objective with high numerical aperture is recommended. The laser light is removed by using a holographic notch filter (in the present example, the Kaiser HS 632,8 holographic notch filter is used) in combination with a conventional long pass filter. As an alternative to this, band pass filters may be employed.

The fluorescence light is detected by using high sensitivity detectors (quantum yield>40%). If the widefield illumination technique is employed, a CCD camera containing a highly sensitive chip must be used. The time resolution of said CCD camera can vary but should be at least 10 ms per image. In the present experiment, the PentaMax camera from Princoten Instruments is used.

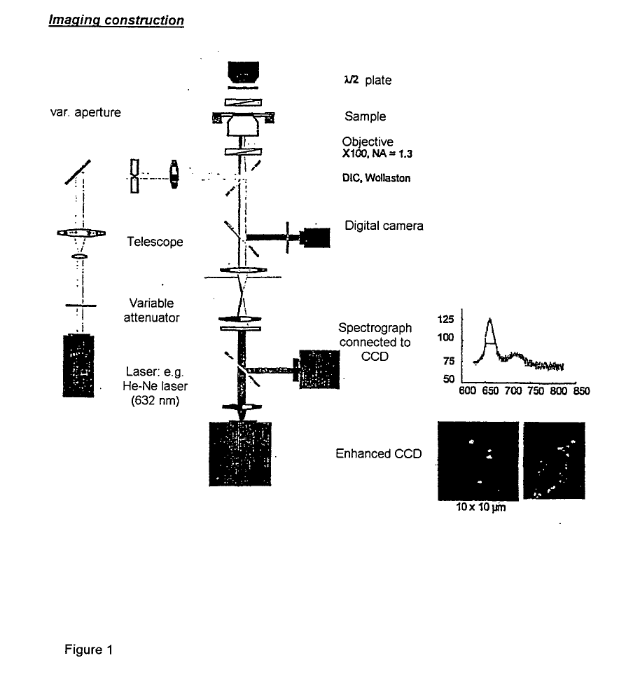

FIG. 1 Apparatus for recording virus trajectories.

The experimental arrangement is depicted diagrammatically. Cell images are recorded in transmitted light mode which is also run using the DIC or phase contrast method in order to be able to label cell components. In confocal mode, three-dimensional transmitted light images are possible. Cell compartments can be labeled with fluorophores and may then also be observed in the fluorescent mode of the confocal variant. Trajectories are recorded in epifluorescence mode which enables recording of 2D projections of trajectories and adjusting the plane of observation in z with the movement of the microscope objective in the direction of the virus particle (piezo-operated feedback mode).

The sample stage can be controlled with respect to pressure and temperature.

The microscope can be operated in transmitted light mode for recording cell images (1), in reflected light mode for detecting fluorescent objects, in particular the location of the virus via fluorescence of the dye label (2), and in reflected light mode for generating fluorescence spectra to detect the dye label (3).

In this connection, the detection is carried out using a commercial digital camera which delivers color images of cells (1), an enhanced CCD camera capable of detecting individual dye molecules (2) or a spectrograph connected to a CCD (3).

The transmitted light mode (1) comprises in any case a simple, commercially common direct light option of an inverted microscope which, however, should also be extendible by special illumination techniques such as phase contrast or differential interference contrast (DIC; indicated here). Thereby, cell components such as the nucleus are emphasized selectively and three-dimensionally in the images.

The reflected light mode (2) primarily operates with widefield illumination which comprises area-to-area imaging (indicated here). Point-to-point imaging which is common in confocal microscopy can be achieved by reducing the variable aperture in the excitation light path. The confocal mode of the microscope is completed by incorporation of a piezo scanner, a detection pinhole and an avalanche photodiode. The confocal technique can produce three-dimensional images of fluorescent objects in the form of halftone images composed of image dots. This is used, in particular, for depicting selectively stainable cell components such as microtubules, actin filaments or intermediate filaments.

Piezo-controlling the objective or the samples axially (not drawn) moves, via a control, the plane of projection of widefield illumination along with the virus particle on the basis of the half-width of the point transfer function of the signal, making it possible to record the trajectory image which is actually two-dimensional in a three-dimensional form within the cell.

The reflected light mode for the recording spectra (3) serves to identify the dye label. It makes possible the recording of spectra both of individual dye molecules and of ensembles of dyes.

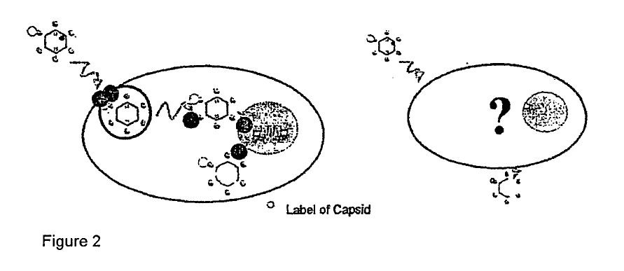

FIG. 2 Representation of viral infection with (left) and without (right) SVT.

SVT tests the mechanisms of action of substances at any site and time of the viral infection and quantifies the action. Conventional methods only see whether or not an infection can be influenced, and are unable to deliver quantitative results. Details of the action of the method can be found in the appendix.

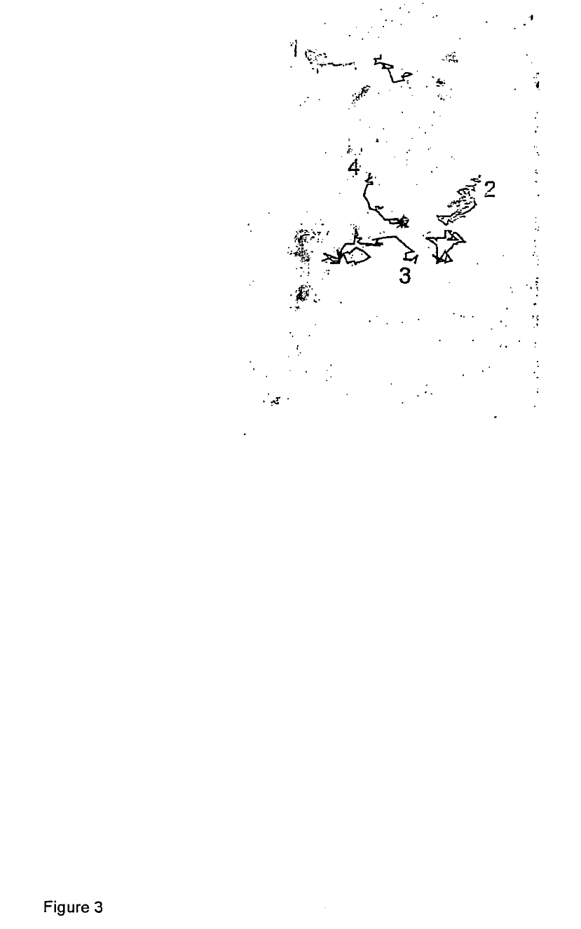

FIG. 3 Trajectories of individual, Cy5-labeled adeno-associated viruses (AAV), which indicate the individual stages of the virus infecting a living cell.

The cell components can be determined from the phase contrast image which is obtained in transmitted light mode using a commercial CCD camera (Nikon Coolpix) mounted on the binocular tube of the microscope. The trajectories are projected into the plane of the sectional cell image recorded. The image contains various, successively recorded traces of viruses. These traces indicate various stages of AAV infection, such as diffusion in aqueous solution (1, 2), touching at the cell membrane (2), penetrating the cell membrane (3), diffusion in the cytoplasm (3, 4) and penetrating the nuclear membrane (4).

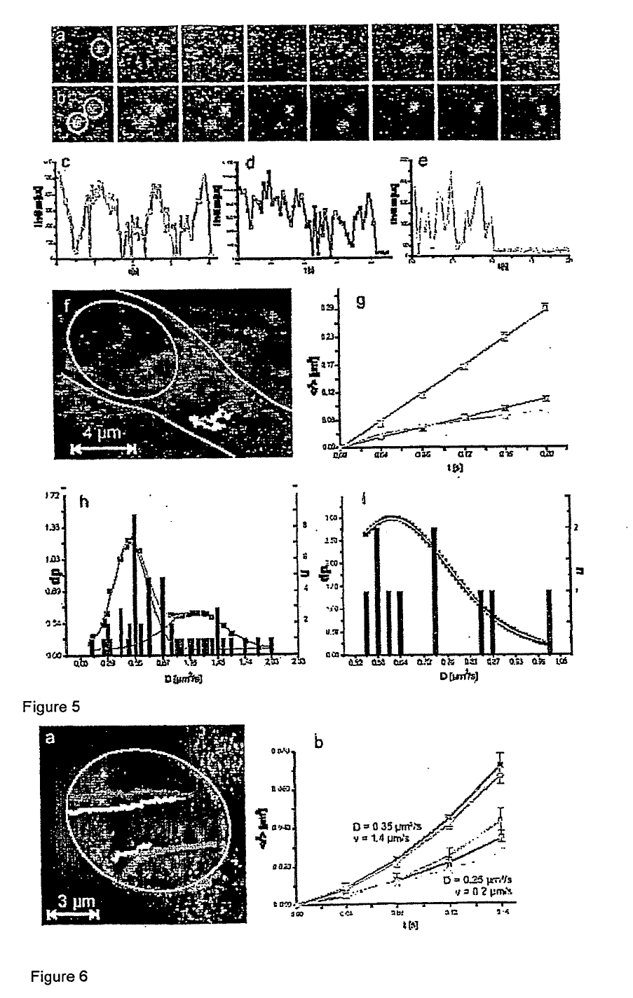

AAV-Cy5 virus solution was added at low concentration (10-⁹ mol l¹ to DMEM cell culture medium containing HeLa cells. An area of 20 im*20 im which contained the image of a cell was recorded in epifluorescence mode of the microscope by using an oil immersion objective (100×Plan-Neofluar, NA 1.3, Zeiss) and a highly sensitive CCD camera (Pentamax, Visitron Systems). When exciting with red laser light (HeNe at 633 nm), the autofluorescence of cells is small enough for making possible the detection of individual fluorophores as Gauss-shaped light spots (fluorescent spots). With the aid of a 2D Gauss fit of the intensity profile, it was possible to determine the positions of the virus molecules to within 40 nm. The trajectories here were recorded with a time resolution of 40 ms. The brightness of the fluorescent spots of a trajectory can be depicted in the form of time tracks (intensity-time diagrams), as shown in FIG. 5. Fluctuations in fluorescence intensity with time are indicated by blinking and photobleaching, i.e. temporary and permanent fading of signals which typically indicates the detection of single molecules. Apart from that, fluctuations occur which indicate movement of the molecule in the z direction and are accompanied by an increase in the half-width of a light spot. Cy5 molecules usually go through 10⁶ photocycles on average in buffer/agarose gel until photobleaching occurs. In the experiments illustrated here, the excitation power is optimized in such a way that enough signal for detecting a fluorophore is possible and, at the same time, the virus molecule can be tracked as long as possible on a single trajectory, until photobleaching occurs. With a detection efficiency of about å=1% and a fluorescence intensity of several hundred counts per spot, it was possible to obtain trajectories of 1-10 seconds on average.

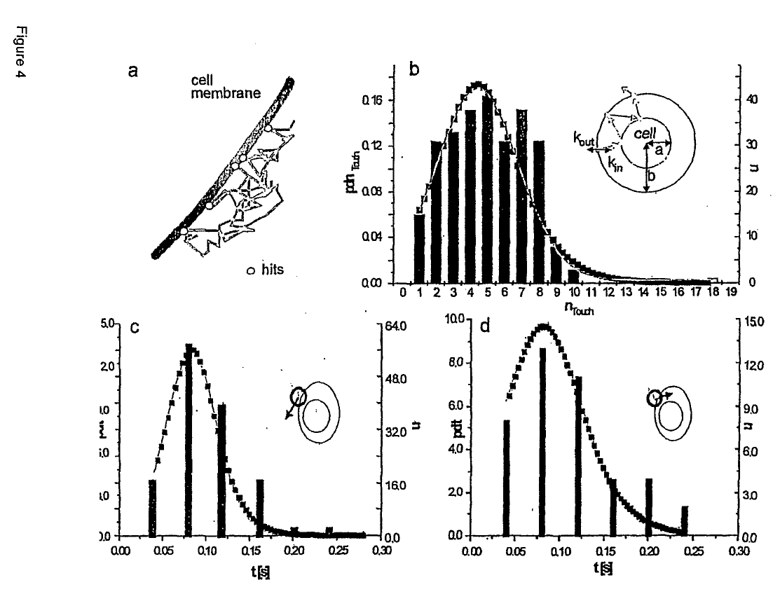

FIG. 4 Uptake of Cy5-labeled AAV into a living HeLa cell

a, “Touching events” at the cell membrane. The trajectory 2 of FIG. 3 is depicted in enlarged form.

It shows 5 touches at the cell surface which are marked by circles and represent short, temporary periods of mobility.

b, The average of such touching series<n_{Touch}> was determined for those viral trajectories which indicated retrodiffusion into the free solution and did not indicate entering of the cell. The statistics are based on 269 trajectories. The frequency distribution is depicted in the form of gray bars.

The accumulated frequency of n_{Touch} was fitted to a sigmoid function. The derivative of this function was normalized and depicted as probability density function pdn_{Touch} (black dots) which was fitted to a Gauss function (red line). The average resulting from said fit was <n_{Touch}>=4.4±3.1.

The model for statistical evaluation of the “touches” on the cell surface is shown as insert in b (Berg, H. C. Random walks in biology (Princeton (N.J.), 1993)). In this model, the cell is considered as a spherical shell with radius r=a. Particles starting at r=b move inward with the rate kᵢₙ and outward with kₒᵤₜ. The probability p for touching the spherical shell is p=kᵢₙ/(kᵢₙ+kₒᵤₜ)=a/b. The average can be determined to <n_{Touch}>=5, if the dimensions for a and b are chosen according to the experimental conditions (a=5 im; b=6 im).

c, d, Distribution of the adsorption times for 137 trajectories which did not indicate entering of the cell (c) and for 42 trajectories which indicated entering (d).

The frequency distributions (bar charts) of adsorption times in virus trajectories are depicted. Probability density functions pdt (black dots) were again formed from the accumulated frequencies (not shown). The Gauss fits (red lines) of these curves produced averages <t>=83±7 ms (c), and <t>=80±11 ms (d), respectively, which showed no significant difference between entering and nonentering of the cell.

FIG. 5 Endosomal migration and diffusion in the cytoplasm.

a, b Two series of fluorescence images show one and two fluorescent spots, respectively. Each of these indicates a single AAV particle. Time resolution: 40 ms.

c-e, Fluorescence time track (1) of these spots from a and b (classification by colors). The plots show the characteristic blinking and single-stage photobleaching which is typical for single molecules. Time track fluctuations indicate diffusive movement in the z direction.

f, Visualization of three trajectories projected on a transmitted light image which was obtained using the conventional microscopic setup as used for fluorescence images. Cell membrane and nucleus are indicated in yellow and were both obtained using the phase contrast of the transmitted light mode.

g, Representation of the mean square displacement as a function of time. The magenta curve results in a diffusion coefficient D=1.5 im²/S which indicates free diffusion of free AAV in the cytoplasm. The green and yellow curves show movements of to endosomes, one with normal diffusion (D=0.55 im²/S green) and the other one with abnormal diffusion (D=0.2 im²/s, á=0.6 yellow).

h, i, Probability density pdD of the diffusion coefficients (determined in analogy to FIG. 4b). The histograms show the distribution of the diffusion coefficients.

The two graphs were obtained from 53 trajectories at pH=7 (h) and 10 trajectories at pH=9 (i). At pH=7, two maxima are found which can be attributed to the free virus (D=1.3 im²/s) and the endosome (D=0.57 im²/s), respectively. The second maximum is similar to the maximum occurring at pH=9 (D=0.64 im²/s) at which only the endosomes can be present in the cell.

FIG. 6 Transport of AAV-Cy5 in the nuclear region.

AAV uses an active transport mechanism (e.g. via the microtubules of the cell) on the route into the nucleus and partly also inside the nucleus. Our microscopy can distinguish this transport mechanism from normal or abnormal diffusion.

a, Five trajectories were projected to the conventionally obtained transmitted light image. The position of the nucleus was again indicated in yellow and determined via phase contrast images. The two trajectories depicted at the bottom run on the same tracks unidirectionally from right to left at different times.

b, Mean square displacement as a function of time. The function profile is parabolic and fulfills the equation <r²>=4Dt+(vt)², with diffusion coefficients D=0.25-0.35 im²/s and rates v=0.2-1.4 im²/s.

1. A method for identifying substances suitable as medicaments for the treatment of viral infections or for testing the effectiveness of said substances, characterized in that individual viruses are labeled with one or more fluorescent dye molecules and the influence of the substance on the viruses or/and their route of infection into the cell and/or inside the cell is determined microscopically at the single virus level via the fluorescence of said dye, after excitation, in comparison with a control sample.

2. The method as claimed in claim 1, characterized in that statistics about a multiplicity of individual viruses and their kinetic/dynamic behavior are compiled which characterize each stage of the infection by the virus and the statistics of the viral infection obtained with the medicaments to be identified and the statistics of the control sample measurement (infection without addition of foreign substance) are compared.

3. The method as claimed in claim 1 or 2, characterized in that the observation is carried out by means of a microscope with a spatial resolution of from 1 to 40 nm in real time and with a time resolution of from 1 to 40 ms.

4. The method as claimed in claim 1, 2 or 3, characterized in that the dye molecule or molecules is/are bound to the virus via N-terminal amino acid residues of proteins.

5. The method as claimed in claim 1, 2 or 3, characterized in that the dye molecule or molecules is/are bound by site-specifically mutated proteins of the virus, for example by means of cysteine.

6. The method as claimed in claim 1, 2, 3, 4 or 5, characterized in that the dye molecule or molecules is/are bound to the viral DNA or to internal proteins.

7. The method as claimed in claim 1, 2 or 3, characterized in that the dye molecule or molecules is/are bound to the virus via selective antibodies, via a specific binding pair or via to cargo systems transported in the virus which may also contain DNA or other drugs.

8. The method as claimed in claim 7, characterized in that specific binding pairs such as, for example, biotin/streptavidin or low molecular weight binding pairs with specific binding regions, are used and preferably small organic components should be bound to viral capsid proteins and the dye.

9. The method as claimed in any of the preceding claims, characterized in that one or more of those fluorescent dyes which exhibits good fluorescence properties, preferably in the long-wave green or in the red spectral region, and can be excited with a large adsorption cross section and which exhibit high fluorescence quantum yield and high photostability are used.

10. The method as claimed in claim 9, characterized in that the dyes used are classical organic fluorophores, such as Cy5 or expressible fluorescent proteins such as green fluorescent protein (GFP) and its mutants or luminescent nanoparticles.

11. The method as claimed in any of the preceding claims, characterized in that at least two dyes which can be distinguished spectrally or by lifetime or by polarization spectroscopy or in (Förster resonance) energy transfer experiments are bound to different positions in the virus, in particular to capsid and DNA.

12. The method as claimed in any of the preceding claims, characterized in that the fluorescent dye is excited by means of a light source, preferably a laser, and particularly preferably a simple helium-neon laser, a frequency-doubled solid state laser or a laser diode.

13. The method as claimed in any of the preceding claims, characterized in that the fluorescent dye is excited via a strong laser, using “two photon excitation”.

14. The method as claimed in any of the preceding claims, characterized in that two or more different fluorescent dyes are excited simultaneously by using various laser lines and a laser with variable frequency.

15. The method as claimed in any of claims 12 to 14, characterized in that the light of the light source is bundled in a microscope or a microscope objective and projected onto the probe.

16. The method as claimed in any of the preceding claims, characterized in that the microscope comprises various modes which make it possible to depict the cell and its components simultaneously or/and independently of detection of the virus.

17. The method as claimed in claim 16, characterized in that the cell is depicted in the transmitted light method using a separate light source and phase contrast, differential interference contrast or polarization contrast techniques are used.

18. The method as claimed in any of the preceding claims, characterized in that two- and/or three-dimensional imaging of the cell or of its components is carried out confocally or by methods using widefield illumination or/and individual organelles or similar subunits of the cell are detected in transmitted light mode or via fluorescence methods using labeled components.

19. The method as claimed in any of the preceding claims, characterized in that the movements of viruses are followed two-dimensionally via excitation in the widefield method and the movement in the axial z dimension is followed by controlling the relative sample/microscope objective movement which automatically tracks the movement of the virus.

20. The method as claimed in any of the preceding claims, in particular as claimed in claim 7 or 11, characterized in that selectively individually labeled subunits in viruses such as, for example, cargo systems transported in the virus or components thereof, individually labeled biomolecules functionally interacting with viruses such as, for example, receptors or nuclear pore complexes, or viruses of a subsequent generation which have been generated by expression in the cell, are monitored or located separately or/and the functional relationship of these units or molecules with one another or with cell components is determined.

21. The method as claimed in any of the preceding claims, characterized in that the sections of the infection biology of the virus, such as adsorption to receptors, endocytosis, diffusion in endosomes, free diffusion, abnormal diffusion, diffusion in inclusions, active transport, for example by means of motor proteins, penetration of the nuclear membrane, colocalization with nuclear pore complexes, breaking up of the virus, insertion of the genome into cellular DNA or production of subsequent generation viruses, are recognized and characterized separately from one another under physiologically relevant conditions.

22. The method as claimed in any of the preceding claims, characterized in that the samples studied are cells which have grown two-dimensionally on a slide, during their infection by the virus.

23. The method as claimed in claim 22, characterized in that the cells are covered by nutrient solution to which fluorescently labeled viruses are added, and fluorescence is observed.

24. The method as claimed in any of the preceding claims, characterized in that the fluorescence signal or fluorescence signals are separated from the excitation light by filters and detected using highly sensitive detectors, preferably one or more CCD cameras or one or more avalanche photodiodes.

25. A method for observing the route or/and mechanism of infection by viruses in cells, for example by viruses which are intended to be used as vectors and/or for gene therapy, characterized in that a method as described in claims 1 to 24 is applied analogously and the virus or its subsequent induced products is/are observed.

26. The method as claimed in claim 25, characterized in that the route of infection of viruses or the route of subsequent induced viral products in cells, which encode as gene shuttles expressible substances which are produced by the host cell as a consequence of the viral infection is observed.
